SEQUENCE LISTING (6 sequences)

SEQ ID NO: 1 - DNA, 20 bp, Artificial Sequence
  PCR primer for IL-10
gcagctgtacccacttccca

SEQ ID NO: 2 - DNA, 17 bp, Artificial Sequence
  PCR primer for IL-10
agaaaacgatgacagcg

SEQ ID NO: 3 - DNA, 20 bp, Artificial Sequence
  PCR primer for actin
atcatgtttgagaccttcaa

SEQ ID NO: 4 - DNA, 20 bp, Artificial Sequence
  PCR primer for actin
catctcttgctcgaagtcca

SEQ ID NO: 5 - DNA, 20 bp, Artificial Sequence
  PCR primer for GAPDH
accaccatggagaaggctgg

SEQ ID NO: 6 - DNA, 20 bp, Artificial Sequence
  PCR primer for GAPDH
ctcagtgtagcccaggatgc

(19) United States
(12) Patent Application Publication
(10) Pub. No.: US 2007/0275441 A1
(43) Pub. Date: Nov. 29, 2007

(54) Ocular Tissue Modification

(75) Inventors: Claire F. Jessup, Mitchell Park (AU); Helen M. Brereton, Bellevue Heights (AU); Douglas J. Coster, Heathpool (AU); Keryn A. Williams, Kingswood (AU)

Correspondence Address: SEED INTELLECTUAL PROPERTY LAW GROUP PLLC, 701 FIFTH AVE, SUITE 5400, SEATTLE, WA, 98104, US

(73) Assignee: The Flinders University of South Australia

(21) Appl. No.: 10/579,995

(22) PCT Filed: Nov. 19, 2004
(86) PCT No.: PCT/AU04/01606

§ 371 Date: May 21, 2007

(30) Foreign Application Priority Data
Nov. 21, 2003 (AU) 2003906440

(51) Int. Cl. C12P 21/00

(52) U.S. Cl. 435/69.6

(57) ABSTRACT

The present invention relates generally to the field of tissue modification, and in particular, but not exclusively, to methods of modifying cells of ocular and particularly corneal tissue to produce immunoglobulins or immunoglobulin fragments. The invention also relates to modified ocular cells and tissues, to expression vectors utilised in such methods, to methods of xeno- and allo-transplantion using the modified tissue and to methods of therapy of ocular diseases and disorders. In a preferred embodiment of the invention there is provided a method of modifying cells of ocular tissue to produce an immunoglobulin or immunoglobulin fragment of interest, which comprises exposing ocular tissue to an effective concentration for transfection of an expression vector comprising a nucleotide sequence encoding for the immunoglobulin or immunoglobulin fragment, for a period sufficient to allow transfection, such that cells of said ocular tissue produce the immunoglobulin or immunoglobulin fragment.

### FIELD OF THE INVENTION

The present invention relates generally to the field of tissue modification, and in particular, but not exclusively, to methods of modifying cells of ocular and particularly corneal tissue to produce immunoglobulins or immunoglobulin fragments. The invention also relates to modified ocular cells and tissues, to expression vectors utilised in such methods, to methods of xeno- and allo-transplantion using the modified tissue and to methods of therapy of ocular diseases and disorders.

### BACKGROUND OF THE INVENTION

There are numerous diseases and disorders that can affect corneal and other ocular tissue and which can, as a result, adversely affect or eliminate vision. For example, allergies, conjunctivitis, corneal infections, Fuchs' dystrophy (deterioration of corneal endothelial cells), varicella-zoster virus, iridocorneal endothelial syndrome, keratoconus, ocular herpes and a number of other conditions, as well as congenital ocular abnormalities, can be responsible for ocular, and particularly corneal, damage or irregularity that may affect vision. In an endeavour to restore sight or improve vision in people suffering from corneal abnormalities it has become particularly common to perform corneal transplant operations where the abnormal corneal tissue is removed and replaced using fine sutures with normal corneal tissue obtained from a donor. Although corneal transplant operations enjoy a high rate of success there are nonetheless some problems that can occur as rejection of the replacement cornea and ocular fibrosis or scarring. Even in the case of a successful corneal transplantation it is necessary for subsequent administration of immunomodulating agents. Non-compliance by the patient with the prescribed dosing regime of immunomodulating agents may give rise to tissue rejection. There is, accordingly, a need for improved means of prolonging corneal or other ocular tissue graft survival and preventing tissue rejection as well as for the provision of approaches for therapy of ocular infection, wounds and fibrosis and for therapy of other ocular disorders, for example.

The cornea is a highly organised group of cells and proteins, which unlike most tissue is clear and does not contain blood vessels to nourish or protect against infection. The cornea receives nutritional supply from tears and the aqueous humor found in the anterior chamber located behind it. The cornea is composed of five basic layers, namely the protective external epithelium, Bowman's layer that is located below the epithelial basement membrane and is composed of collagen fibres, the stroma that consists primarily of water and collagen and is located beneath Bowman's layer, and Descemet's membrane located beneath the stroma, which is composed of collagen fibres produced by the endothelial cells located in the lower endothelium. The endothelial cells are essential in maintaining clarity of the cornea by removing excess fluid from the stroma.

Irreversible immunological rejection is the major cause of human corneal graft failure (1), despite the immunologically privileged nature of the eye (2). The histological correlates of rejection include local upregulation of major histocompatability complex and adhesion molecules, an influx of mononuclear cells into the cornea and anterior chamber, and local production of some inflammatory cytokines (3-7). The major target of corneal graft rejection is the corneal endothelium. Human (but not rodent) corneal endothelium is essentially amitotic (8), so that damage to the monolayer during graft rejection cannot be repaired.

Gene therapy has the potential to influence an allograft response through local production of immunoglobulins or immunoglobulin fragments within transplanted donor tissue. The local production of immunoglobulins or immunoglobulin fragments within ocular, and particularly corneal cells, may also provide or enhance therapies for ocular infections and wounds. The present inventors consider that the cornea may be particularly amenable to the improved allograft methods of the present invention because of its small size, which may allow modification of the whole tissue, and because of the ease with which a donor cornea may be manipulated in vitro and stored for a considerable period (for example, up to 28 days) prior to transplantation. The anatomical location and clarity of the cornea allow in vivo assessment of the entire graft in the post-operative period and loss of function is easy to detect. Furthermore, the cornea and anterior chamber are at least partially immunologically privileged sites (2), which may allow the use of otherwise immunogenic or pro-inflammatory vectors.

Gene therapy approaches to production of immunoglobulins or immunoglobulin fragments within ocular cells also offer potential for treatment of ocular disorders and wounds or injury as well as prophylactic approaches following ocular wounds or injury, to prevent or minimise the severity of infection.

Surprisingly the present inventors have now demonstrated that immunoglobulins or immunoglobulin fragments may be produced in ocular tissue by gene therapy approaches and that subject to the selection of suitable immunoglobulins or immunoglobulin fragments these approaches can offer a therapeutic benefit.

### SUMMARY OF THE INVENTION

According to one embodiment of the present invention there is provided a method of modifying cells of ocular tissue to produce an immunoglobulin or immunoglobulin fragment of interest, which comprises exposing ocular tissue to an effective concentration for transfection of an expression vector comprising a nucleotide sequence encoding for the immunoglobulin or immunoglobulin fragment, for a period sufficient to allow transfection, such that cells of said ocular tissue produce the immunoglobulin or immunoglobulin fragment.

According to another embodiment of the present invention there is provided an ocular tissue comprising cells modified to express an immunoglobulin or immunoglobulin fragment.

The nucleotide sequence utilized in the methods of the invention can be any type of nucleic acid, e.g., DNA or RNA, preferably DNA such as gDNA or most preferably cDNA. The nucleic acid can be a full-length gene or an active portion of the gene. It may also comprise more than one discrete sequence, which sequences are designed to be separately translated within the cell in which they subsequently associate.

In another embodiment of the invention there is provided a harvested ocular tissue comprising cells modified to express an immunoglobulin or immunoglobulin fragment, wherein modification is by exposing ocular tissue to an effective concentration for transfection of an expression vector comprising a nucleotide sequence encoding for the immunoglobulin or immunoglobulin fragment, for a period sufficient to allow transfection.

In a further embodiment of the invention there is provided a method of improving ocular graft healing and/or prolonging graft survival comprising exposing ocular tissue to an effective concentration for transfection of an expression vector comprising a nucleotide sequence encoding for an immunoglobulin or immunoglobulin fragment specific for an agent or agents associated with ocular graft healing and/or rejection, for a period sufficient to allow transfection such that cells of said ocular tissue will express the immunoglobulin or immunoglobulin fragment, harvesting said tissue from a donor and transplanting the ocular tissue to an eye of a recipient.

In a still further embodiment of the invention there is provided an expression vector for use in modifying ocular tissue to express an immunoglobulin or immunoglobulin fragment comprising a nucleotide sequence encoding for the immunoglobulin or immunoglobulin fragment.

In preferred embodiments of the invention the ocular tissue is harvested from a mammal, particularly preferably from a human. Preferably the recipient of transplanted ocular tissue is a mammal, particularly preferably a human. In a particularly preferred embodiment of the invention ocular tissue is harvested from a human and transplanted to another human recipient.

According to another embodiment of the present invention there is provided a method of treatment and/or prophylaxis of an ocular disorder which comprises introducing into the eye of a mammalian patient an effective concentration for transfection into an ocular tissue of an expression vector comprising a nucleotide sequence encoding for an immunoglobulin or immunoglobulin fragment specific for an agent associated with the disorder, for a period sufficient to allow transfection such that cells of said ocular tissue will produce the immunoglobulin or immunoglobulin fragment.

Preferably the ocular tissue is pupil, iris, vitreous, macula, retina, sclera, lens, choroid, limbal tissue, conjunctiva or corneal tissue. Preferably the ocular tissue is corneal tissue or limbal tissue. The use of limbal tissue is described for example by Swift et al 1996; Williams et al 1995; Henderson et al 2001; and Mills et al 2002.

In another embodiment of the invention the expression vector or another expression vector transfected into the ocular tissue cells comprises a nucleotide sequence encoding for an active agent. Preferably the active agent is a peptide hormone, a cytokine or an analogue thereof. In a preferred embodiment of the invention the cytokine is an interleukin, an interferon or a growth factor, or an analogue thereof. In preferred embodiments of the invention the cytokine is selected from the interleukins including IL-10, IL-4, the P-40 component of IL-12 or from Bcl2, interferon Gamma, interferon Alpha and TGF Beta.

Preferably at least 5%, preferably at least 10%, more preferably at least 20%, particularly preferably at least 30% or at least 50% and most particularly preferably at least 70% of cells in the ocular tissue are modified by the methods of the invention. In a preferred embodiment of the invention the modified ocular tissue cells, preferably corneal tissue cells, are epithelial cells, stroma cells and/or endothelial cells. Particularly preferably, the modified cells are endothelial cells. In a preferred embodiment of the invention 5%, preferably at least 10%, more preferably at least 20%, particularly preferably at least 30% or at least 50% and most particularly preferably at least 70% of corneal endothelial cells in a sample of corneal endothelial cells are modified by methods according to the invention.

In preferred embodiments of the invention the expression vector is a viral, bacterial or plasmid vector. In particularly preferred embodiments of the invention the expression vector is an adeno-associated viral vector, an adenoviral vector, a lentiviral vector or herpes viral (eg. herpes simplex) vector. In another embodiment of the invention the expression vector is a non-live vector such as a liposomal vector.

Preferably the effective concentration for transfection is between about 1×10⁵ to 1×10¹⁰ plaque forming units (pfu) per gm. tissue (eg. cornea). Particularly preferably the effective concentration for transfection is between about 5×10⁵ to 5×10⁸ pfu/gm. tissue, more particularly preferably between about 2×10⁶ and about 1×10⁸ pfu/gm. tissue.

In a preferred embodiment of the invention the period sufficient to allow transfection is between about 1 minute and about 48 hours, particularly preferably between about 10 minutes and 24 hours, more particularly preferably between about 30 minutes and 6 hours and most particularly preferably between about 1 hour and about 3 hours.

In another preferred embodiment of the invention the expression vector comprises DNA sequences encoding for two or more immunoglobulins and/or immunoglobulin fragments (optionally with an active agent) and/or two or more active agents.

### BRIEF DESCRIPTION OF THE DRAWINGS

The invention will be further described, by way of example only with reference to the drawings, wherein:

FIG. 1. Autoradiographs showing uptake of 3H thymidine in organ-cultured ovine corneal endothelium, 3 days after deliberate injury. FIG. 1(A) shows uptake in endothelial cells (arrowed) at margins of deliberate injury; magnification ×32; FIG. 1(B) shows high power view showing mitotic figure (arrowed); magnification ×128.

FIG. 2. Effect of viral concentration and incubation time with virus on transfection efficiency of the adenoviral vector Ad-lacZ for ovine corneal endothelium, and stability of expression of -galactosidase in endothelial cells of organ-cultured ovine corneas. In each instance, reporter gene expression was quantified by counting-galactosidase-positive cells in three representative areas per cornea. FIG. 2(A) shows concentrations of Ad-lacZ from 6.6×10² to 6.6×10⁸ pfu per cornea were used to transfect ovine corneas in vitro under otherwise identical conditions. Corneas were harvested 48 hours later. Each bar represents the mean percentage positive cells ±SD of counts from 3 to 6 corneas. FIG. 2(B) shows ovine corneas were incubated with Ad-lacZ at 6.6×10⁶ and 6.6×10⁷ pfu per cornea for 0.5-2.0 h. Corneas were harvested 48 hours later. Each bar represents the mean percentage of positive cells ±SD from 3 organ-cultured corneas. FIG. 2(C) shows duration of expression of -galactosidase in organ-cultured ovine corneas after transfection with 6.6×10⁶ Ad-lacz pfu per cornea for 2 hours. Corneas were harvested at the indicated time-points. Each point represents the mean percentage positive cells ±SD of 3-14 corneas.

FIG. 3. Agarose 1.5% gel showing product for IL-10 and GAPDH in sheep corneas transfected with Ad-IL-10 under optimal conditions and organ-cultured for 21 days prior to RNA extraction and RT-PCR. Dilutions of cDNA at 1/1, 1/10 and 1/100 dilutions were run in duplicate lanes. Lanes marked no DNA represent controls in which water replaced cDNA.

FIG. 4. Outcome of gene-modified penetrating corneal allografts in sheep: FIG. 4(A) shows rejecting unmodified allograft, day 29 post graft; FIG. 4(B) shows surviving IL-10-modified allograft, day 190 post-graft.

FIG. 5. Rat thymocyte binding activity of infected rat corneal supernatants (day 4 post infection). Activity was detected in the supernatant of corneas infected with anti-CD4 single-chain antibody fragment-encoding adenovirus (AdCD4; black histogram) but not in corneas infected with enhanced green fluorescent protein-encoding adenovirus (AdGFP; grey histogram).

FIG. 6. Effect of anti-CD4 monomeric single-chain antibody fragment on the mixed lymphocyte reaction. Anti-CD4 single chain antibody fragment (20mer monomer) or vehicle (monomer vehicle) was added to wells containing 2×10⁵ F344 responders and 4×10⁵ WF stimulators. Bars represent means of triplicate wells. Error bars represent ±SD. Data was transformed (log₁₀) and analysed by 2-way ANOVA, followed by multicomparison tests on simple main effects. *P<0.05 was considered significant (adjusted by Bonferroni).

FIG. 7. Effect of anti-CD4 dimeric single-chain antibody fragment on the mixed lymphocyte reaction. Anti-CD4 single chain antibody fragment (20mer dimer or 11mer dimer) or vehicle (dimer vehicle) was added to wells containing 2×10⁵ F344 responders and 4×10⁵ WF stimulators. Bars represent means of triplicate wells. Error bars represent 1 SD. Data was transformed (log₁₀) and analysed by 2-way ANOVA, followed by multicomparison tests on simple main effects. *P<0.05 was considered significant (adjusted by Bonferroni).

FIG. 8. Detection of transgene mRNA in transduced rat corneas by RT-PCR. cDNA encoding reporter gene (GFP; 300 bp) or single chain antibody fragment (scFv; 1 kb) were found in corneas transduced with anti-CD4 scFv-encoding adenovirus (Ad-CD4). CDNA encoding reporter gene only was found in corneas transduced with a control virus (AdGFP)

FIG. 9. Detection of single-chain antibody fragment protein by SDS-PAGE and Western Biot. His-tagged protein of the correct size (arrows; 26 kDa) was detected anti-histidine antibody in affinity purified supernatant from HEK-293 cells transduced with anti-CD4 single-chain antibody fragment-encoding adenovirus.

### SEQUENCE LISTINGS

The subject specification contains DNA sequence information prepared using the programme PatentIn Version 3.1. Each DNA sequence is identified in the sequence listing by the numeric indicator <201> followed by the sequence identifier (eg. <210>1, <210>2, etc). The length, type of sequence (prt, etc) and source for each sequence is indicated by information provided in the numeric indicator fields <211>, <212> and <213>, respectively. DNA sequences referred to in the specification are identified by the indicator SEQ ID NO: followed by the sequence identifier (eg. SEQ ID NO:1, SEQ ID NO:2, etc.). The sequence identifier referred to in the specification correlates to the information provided in numeric indicator field <400> in the sequence listing, which is followed by the sequence identifier (eg. <400>1, <400>2, etc). That is SEQ ID NO:1 as detailed in the specification correlates to the sequence indicated as <400>1 in the sequence listing.
SEQ ID NO. 1 shows the cDNA sequence of a PCR primer for IL-10.
SEQ ID NO. 2 shows the cDNA sequence of a PCR primer for IL-10.
SEQ ID NO. 3 shows the cDNA sequence of a PCR primer for actin.
SEQ ID NO. 4 shows the cDNA sequence of a PCR primer for actin.
SEQ ID NO. 5 shows the cDNA sequence of a PCR primer for GAPDH.
SEQ ID NO. 6 shows the cDNA sequence of a PCR primer for GAPDH.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that that prior art forms part of the common general knowledge in Australia.

As indicated above the present inventors have devised an approach for treating ocular disorders and demonstrated its efficacy in animal models. In the specifically exemplified embodiments of the present invention harvested corneal tissue has been modified to produce immunoglobulin fragments having the effect of prolonging corneal graft survival once implanted into a recipient, relative to the expected survival time in the case where no nucleotide sequence encoding immunoglobulin fragments is administered. However, the present invention has considerably broader application than simply its use in association with corneal allo- or xeno-transplantation techniques. Methods according to the present invention may be adopted for treatment of other ocular disorders such as, for example, the treatment of ocular wounds, infections or fibrosis or of other ocular diseases such as glaucoma, keratoconus, corneal dystrophies, corneal infections, tumours of the eye, proliferative lesions, pterygium and inflammatory disorders of the eye including Stevens-Johnson syndrome and mucous pemphigoid. Reference to the term “treatment” is intended to include both therapeutic and prophylactic treatments.

By the term “harvested” it is intended to convey that the particular ocular tissue has been removed from the eye of a donor animal. It may then be maintained under appropriate tissue culture conditions or may be transplanted to the eye of another animal from the same or different species. Modification of cells of harvested tissue may take place before the tissue is removed from the donor animal, as it is being removed from the donor animal, while it is in tissue culture or indeed once it has been transplanted, for example. An initial site of transplantation need not necessarily be the final intended locus of the tissue. For example, the tissue may be removed from the donor animal and cultured in the eye of a host animal before being removed and transplanted to the final recipient animal.

One aspect of the invention relates to methods of modifying cells of ocular tissue to produce an immunoglobulin or immunoglobulin fragment. The immunoglobulin or immunoglobulin fragment will comprise a preferably monoclonal antibody or antibody fragment raised to have specificity against an agent or agents such that it is suitable for the intended method of therapy or prophylaxis in which it is to be used. Depending upon the intended outcome of the treatment or prophylactic methods the immunoglobulin or immunoglobulin fragment may be specific for an agent or agents associated with ocular graft healing and/or rejection or associated with an ocular disorder. For example, immunoglobulins or fragments thereof to be produced in modified ocular cells according to the invention may have specificity for any of a large number of inflammatory molecules, receptors for inflammatory molecules or microbial agents capable of infecting the eye. Binding of the immunoglobulin or immunoglobulin fragment to such agents will interact with the agent to eliminate or at least substantially reduce the adverse action of this agent in relation to graft rejection or the ocular disorder concerned. Suitable target antigens include, but are not limited to, an MHC molecule including MHC molecules that are class I- or class II-restricted, a co-stimulatory molecule (eg., CD80, CD86 and CD152), an adhesion molecule (eg. CD11b/e, CD18, CD54 and CD62L), a receptor-associated molecule (eg., CD3, CD4, CD8, CD28, CD40, CD40L, ICOS, PD-1, BTLA and CTLA4), a cytokine receptor (eg., the interleukin 2 receptor (IL-2R), or a subunit thereof such as CD25, and the interferon γ receptor (IFN-γR) and a viral surface antigen (eg., gD2 and gB2 antigen of herpes simplex virus or a surface antigen of the herpes virus causing herpetic-keratitis). A further microbial target is one or more of the surface antigens of Acanthamosha Nucleotide sequences encoding for suitable immunoglobulins and immunoglobulin fragments are available in public access databases, and may be synthesised or otherwise obtained by conventional approaches, as for example further explained in Sambrook & Russell, Molecular Cloning: A laboratory manual, 3rd Edition, 2001, Cold Spring Harbour Laboratory Press, New York, the entire contents of which are included herein in their entirety by way of reference. Examples of suitable immunoglobulin fragments i=we single-chain variable-domain antibody fragments (scFv), Fab fragments, diabodies, minibodies, tribodies and dimers and aggregates thereof. It is also possible to modify cells of ocular tissue to produce an active agent or agents in addition to one or more immunoglobulin or immunoglobulin fragment. As these active agents are to be expressed in the ocular tissue cells as a result of gene transfer, the active agents will of course constitute peptides, polypeptides or proteins, encoded for by nucleotide, particularly DNA sequences. Collectively, such active agents, regardless of the peptide sequence, may be referred to herein as “peptides”. The active agents according to the invention may for example constitute naturally occurring or synthetic peptide hormones, cytokines or analogues thereof. By use of the term “analogue” it is intended to embrace modified forms of naturally occurring or synthetic peptide hormones or cytokines (having physiological activity that may for example be modified relative to the molecule upon which they are based) that are altered by the addition, deletion or substitution of single or multiple amino acids.

Examples of active agents that may be expressed by the methods and vectors according to the present invention include peptide hormones and cytokines and analogues thereof that may for example have immunomodulatory, anti-infective, tissue regeneration, wound healing or fibrosis reduction activity. Cytokines that may be adopted in the present invention include those selected from the interleukins, the interferons and the growth factors as well as analogues thereof. Specific examples of cytokines that may be adopted include IL-10, IL-4, the P-40 component of IL-12, Bcl2, interferon gamma, interferon alpha and TGF beta. It is to be understood however that these specific cytokines are mentioned as active agents by way of example only, and that other peptide agents with useful activity may equally be adopted.

The ocular tissue to be modified according to the present invention may have been harvested from a donor, usually a donor mammal. Preferably the donor will be selected from the same species as the transplant recipient and generally from a donor having matching tissue and/or blood types as the intended recipient, as well understood in the art. There may, however, be circumstances, such as if there is insufficient donor organ supply from members of the same species (allo-transplantation), where ocular tissue is harvested from a donor member of another species (xeno-transplantation). In the case of xeno-transplantation the tissue donor may be an animal that has been genetically modified to remove or reduce the impact of species specific immunogenic differences. It may also be possible in future for ocular tissue to be produced by organ culture techniques, which can then similarly be harvested for modification by methods according to the present invention. Mammals from which ocular tissue may be harvested and/or to which ocular tissue may be transplanted include, but are not limited to, humans, farm animals including cattle, sheep, goats, pigs, horses, etc.; captive wild animals including lions, tigers, deer, chimpanzees, apes, gorillas, baboons, etc.; domestic animals such as cats and dogs, etc, or laboratory animals such as rabbits, mice, guinea pigs, rats and the like. Preferably the ocular tissue is harvested from a human donor for transplant to a human recipient. In the case of human ocular tissue donors, the donor will generally be a person registered as an organ donor who has met an untimely death, and whose ocular tissue of interest is in good condition. In the case of animal donors, the animal may be sacrificed in order to harvest the ocular tissue or may in fact be sacrificed for other purposes such that the ocular tissue becomes available.

The ocular tissue will preferably be obtained from the donor within a relatively short period post mortem, preferably within three to four hours and particularly preferably within the first hour. The conditions under which the ocular tissue should be removed from the donor and maintained prior to modification are a matter of routine and are well understood by persons skilled in the art. Naturally, the use of a suitable tissue culture media is required to maintain the tissue in a healthy state prior to modification and transplantation. Preferably modification of the tissue will be conducted within a matter of a few has from harvesting of the tissue, although it is possible to Sin ocular, and particularly corneal, tissue under tissue culture conditions for up to about 28 days.

In other aspects of the invention the ocular tissue may be modified in situ within the patient by topical administration of the expression vector to the eye generally or to specific ocular tissue by routes of administration and in conjunction with conventional carriers and/or excipients, as discussed further below. Such approaches may be adopted in endeavors to treat ocular disorders or, for example, to prevent complications or infections that could result from ocular wounds or injury.

The ocular tissue modified according to the invention may be selected from a variety of ocular tissue types such as pupil, iris, vitreous, macula, retina, sclera, lens, choroid, limbal tissue, conjunctiva or corneal tissue. Preferably the ocular tissue is corneal tissue.

The expression vector according to the present invention may constitute any of a wide variety of already known or even as yet unidentified types of expression vector, such as viral, bacterial or plasmid expression vector systems or non-live expression vectors such as liposomal expression vectors as are well known in the art. Genes may be delivered to ocular tissue either in vivo or ex vivo. To date, in vivo delivery has been achieved with most success by use of live viral carriers in particular adenovirus, lentiviruses (including HIV) and herpes simplex virus (for a review see Borras; 2003, Exp. Eye Res. 76 643-52). This review also contemplates use of electric pulses and direct injection of naked DNA. Other techniques which have been used for in vivo delivery include direct injection in a lipid vehicle (Mandava et al Invest. Opthalmol. Vis. Sci. 2002 43 3338-48)), pegylated immunoliposomes (Zhang et al Mol. Vis. 2003 9 465-72) and iontophoresis (Berdugo et al Antisense nucleic acid drug. dev. 2003 13 107-14). These and additional techniques have also been reviewed by Kurz (Ophthalmol Clin. North Am 2002 15 405-10).

Techniques which have been used for ex vivo delivery include all the above along with techniques not currently suitable for use in a living animal including particle bombardment (Shestopolov et al, Exp. Eye Res. 2002 74 639-49), and various chemical-based procedures including cationic liposomes. These are reviewed by Grobhans Funct. Integr. Genomics 2000 1 142-5 and by Wolf and Jenkins Int. J. Oncol. 2002 21 461-68. General laboratory techniques for transfection of cells and tissues can also be found in Sambrook, & Russell. Molecular Cloning: A Laboratory Manual 3rd Edition 2001. Cold Spring Harbor Laboratory Press, New York.

Naturally, the expression vector adopted must be one that can transfect, and result in protein production in the desired ocular tissue cells and particularly in cells of the corneal epithelium, stroma and/or endothelium. Preferably, expression of the active agent, through infection by the expression vector, is within the endothelial cells and particularly preferably the level of infection of these cells with the selected expression vector is such that active ingredient expression is demonstrated in at least 5%, preferably at least 10%, more preferably at least 20%, particularly preferably at least 30% or at least 50% and most particularly preferably at least 70% in a sample of corneal endothelial cells. The expression vector selected will of course include all of the features required for production of protein in a mammalian cell. For example, preferred expression vectors will comprise a molecular chimera containing the coding sequences of the immunoglobulin or immunoglobulin fragment and optional active agent or agents selected, an appropriate polyadenylation signal for a mammalian gene (i.e. a polyadenylation signal which will function in a mammalian gene), and suitable enhancers and promoter sequences in the correct orientation.

In mammalian cells, normally two DNA sequences are required for the complete and efficient transcriptional regulation of genes that encode messenger RNAs in mammalian cells: promoters and enhancers. Promoters are located immediately upstream (5′ from the start side of transcription. Promoter sequences are required for accurate and efficient initiation of transcription. Different promoters reveal a common pattern or organisation. A typical promoter includes an AT-rich region called a TATA box (which is located approximately 30 base pairs 5′ to the transcription initiation start site) and one or more upstream promoter elements (UPE). The UPEs are a principle target for the interaction with sequence-specific nuclear transcription factors. The activity of promoter sequences is modulated by other sequences called enhancers. The enhancer sequence may be a great distance from the promoter in either upstream (5′) or downstream (3′) position. Hence, enhancers operate in an orientation- and position-independent manner. However, based on similar structural organisation and function that may be interchanged the absolute distinction between promoters and enhancers is somewhat arbitrary. Enhancers increase the rate of transcription from the promoter sequence. The necessary machinery required for cellular expression of the nucleotide sequence must of course be located in the appropriate orientation with regard to the nucleotide sequence (preferably DNA) inserted into the expression vector by the use of routine molecular biology techniques, for example as further explained in Ausubel et al. (1987) in: Current Protocols in Molecular Biology, Wyle Interscience (ISBN 047150338) the disclosure of which is incorporated herein by reference in its entirety. Insertion of steroid-inducible promotor/enhancer combinations will allow steroid-inducible control of transgene expression. Also mentioned by way of reference in relation to preparation of expression vectors, the disclosure of which is included herein by reference is He et al., “A simplified system for generating recombinant adenovirus”, Proc. Nat. Acad. Sci. (1998) 95: 2509-2514. The expression vector can appropriately include a suitable nuclear localisation signal and may be propagated in any permissive cell line. Permissive cell lines, mentioned by way of example only, include E1A and E1B trans-complementing 293 cells. Other cell lines will equally be useful for propagation of expression vectors according to the invention, as would clearly be understood by persons skilled in the art.

The exposure of ocular tissue to expression vectors according to the invention will be in a manner that will allow transfection by the expression vector of the tissue cells. The exposure of the tissue to the expression vector can simply be by including the expression vector into the tissue culture media, or by direct (topical) or injection administration to the eye optionally in conjunction with one or more pharmaceutically acceptable carriers and/or excipients (as for example disclosed in Remington's Pharmaceutical Sciences, Mack Publishing Co., Easton, Pa., USA, the disclosure of which is included herein in its entirety by way of reference). For administration to the eye the expression vector may be formulated as a cream, paste or tincture, as drops or in an injectable form, for example.

Other means of exposure such as via direct injection of the expression vectors into the ocular tissue or via high velocity bombardment may also be adopted, although care should be taken to avoid damage to the tissue. To ensure adequate levels of transfection of ocular cells with the expression vector it is necessary for an effective concentration for transfection of the expression vector to be utilised. For example, concentrations of between about 1×10⁵ to 1×10¹⁰ plaque forming units (pfu) per gm. tissue (eg. cornea) may be used. Particularly preferably the effective concentration for transfection is between about 5×10⁵ to 5×10⁸ pfu/gm. tissue, more particularly preferably between about 2×10⁶ and about 1×10⁸ pfu/gm. tissue.

The invention will now be further described with reference to the following non-limiting examples.

### EXAMPLE 1

### Transplantation in the Outbred Sheep of Corneal Tissue Modified by Gene Therapy Using Various Expression Vectors to Produce the Immunomodulatory Cytokine IL-10

The inventors selected a model of orthotopic corneal transplantation in the outbred sheep, a relevant preclinical model in which unmodified corneal allografts undergo rejection at three weeks post-operatively in a manner that is very similar at a clinical level to human corneal graft rejection (9). Adenoviral vectors have already been shown to be capable of transferring reporter genes into corneal endothelium of various species (10-13) and the use of liposomal agents has also previously been explored (14, 15). Given that the mitotic potential of sheep corneal endothelium was unknown, the replicative capacity of this tissue was first examined, to allow an informed choice of the vector for gene therapy to be made. The immunomodulatory cytokine, IL-10, which down-regulates cell-mediated immune responses under some circumstances (16, 17), was chosen as the candidate gene product for regulation of allograft rejection by ex vivo gene therapy.

Materials and Methods

Ovine corneal organ-culture. Fresh sheep eyes obtained within 3 hr of donor death from a local abattoir (Lobethal Abattoirs, Lobethal, S A, Australia) were decontaminated for 3 min in 10% w/v povidone-iodine (Faulding Pharmaceuticals, Salisbury, S A, Australia) and underwent two washes by immersion in sterile 0.9% w/v NaCl. A limbal incision was made with a scalpel blade and the cornea with a 2 mm scleral rim was removed with corneal scissors. Corneas were organ-cultured in 15 ml complete medium (HEPES-buffered RPMI medium (ICN, Costa Mesa, Calif., USA) supplemented with 10% v/v heat-inactivated (56 C, 30 min) fetal calf serum (FCS), 100 IU/ml penicillin, 100 g/ml streptomycin, 2.5 g/ml amphotericin B and 2 mM L-glutamine (all from Gibco BRL, Gaithersburg, Md., USA) at 32 C in air for up to 28 days. Medium was changed twice weekly.

Evaluation of the mitotic potential of ovine corneal endothelium. A 4 mm long central cross-shaped defect was produced with a 27 gauge needle on endothelial monolayers of fresh ovine corneas. Corneas were then placed in sterile shallow wells, endothelium facing upward. 500 l complete medium containing 25 Ci 6-³H thymidine (TRA61; Amersham, Little Chalfont, Buckinghamshire, UK) was placed in the corneal cup for 5 hr at 32 C. The solution was then diluted to a total volume of 3 ml with complete medium containing no isotope and to 10 ml total after 24 hr. After 3 days, corneas were harvested, fixed in 3:1 absolute ethanol:glacial acetic acid at room temperature for 24 hr and transferred to 70% ethanol for a further 24 hr. Corneal endothelium was removed by blunt dissection through the stroma, mounted on gelatin-coated slides and air-dried for 2 hr. Flat-mounts were coated with LM-1 photographic autoradiography emulsion (Amersham, Little Chalfont, Buckinghamshire, UK), exposed at 4 C for 4 weeks and processed according to the manufacturer's protocol. The flat-mounts were stained with Giemsa and mounted in Depex (BDH Chemicals, Kilsyth, VIC, Australia). As negative controls, corneas were injured and incubated in ³H thymidine-free medium, and uninjured corneas were incubated with and without 3H thymidine. Corneal epithelial flat-mounts prepared from corneas incubated as above with the epithelial surface in contact with tritiated thymidine-containing medium were used as a positive control.

Transfection of ovine corneal endothelium with adenoviral vectors. The replication deficient E1-, E3-deleted adenovirus type 5 vectors encoding E. coli lacZ under the transcriptional control of the CMV promoter (Ad-lacZ), or containing an empty plasmid (Ad-mock), or encoding full-length ovine IL-10 (Ad-IL-10) or P-40 subunit of IL-12 (Ad-P40-IL-12) (cDNA sequence provided by Dr S. Swinburn, Haematology Department, Flinders Medical Centre, South Australia) where prepared following the approach as described in Hu et al. as referenced above. cDNA sequences for these species are available on public databases. The Ad-lacZ construct contained a nuclear localization signal. Vectors were propagated in E1A, E1B trans-complementing 293 cells following standard protocols (18-20). In order to determine optimal viral concentration for infection of corneal endothelial cells, corneas were infected with concentrations of Ad-lacZ ranging from 6.6×10²-6.6×10⁸ plaque forming units (pfu) per cornea in complete medium. Control corneas were uninfected or similarly infected with Ad-mock. Optimal infection time was determined by incubation of the corneas with 6.6×10⁶ and 6.6×10⁷ pfu Ad-lacZ per cornea for 0.5, 1, 1.5 and 2 hours; the vector was then diluted out and the corneas were re-incubated for a further 48 hr in 15 ml complete medium. To examine duration of reporter gene expression, corneas infected with 6.6×10⁷ pfu per cornea for 2 hours at room temperature were organ-cultured for 2 days (n=14), 3 days (n=6), 6 days (n=6), 7 days (n==1), 10 days (n=5), 13 days (n=5), 14 days (n=1), 16 days (n=3), 21 days (n=4), and 28 days (n=3). After incubation, all corneas were processed for lacZ reporter gene expression.

Detection of lacZ reporter gene expression. Prior to processing, corneas were fixed in 2.5% formaldehyde and 0.25% glutaraldehyde in Dulbecco's A phosphate-buffered saline (PBS) for 15 min on ice followed by two 15 min washes in PBS on ice to inactivate the viral vector and inhibit endogenous-galactosidase (21). Expression of E. coli galactosidase was detected using 2.5 ml/cornea of a solution of 1 mg/ml 5-bromo-4-chloro-3-indoxyl-D-galactoside (ICN, Costa Mesa, Calif., USA), N-dimethylformamide (BDH Chemicals, Kilsyth, VIC, Australia), 2 mM MgCl₂, 5 mM K₄Fe(CN)₆, 5 mM K₃Fe(CN)₆ in PBS-2 (16 mM Na₂HPO₄, 4 mM NaH₂PO₄.2H₂O, 120 mM NaCl), pH 7.0 at 32 C for 18 hr in the dark. After a 10 min wash with 20 ml water per cornea, a modified silver stain to stain endothelial cell boundaries was performed by application of 1% w/v AgNO₃ for 1 min and subsequent exposure to light (22). The endothelium was surgically removed using a 23 gauge needle and toothed forceps, and mounted in Kaiser's glycerol jelly (12.5% w/v gelatin, 87.5% v/v glycerin) on chrome-alum subbed slides. To detect E. coli-galactosidase in 293 cells, cells were washed twice with PBS, fixed on ice with 0.25% glutaraldehyde in PBS for 5 min and washed twice with ice-cold PBS. Staining was then performed as described above.

Quantification of lacZ expression. To quantify the number of cells expressing the reporter gene, corneal endothelial flat-mounts were examined by light microscopy and photographed on 35 mm slide film at standard magnifications. The slides were projected at a standard distance and magnification. Total numbers of endothelial cells and lacZ positive cells were counted within frames of known dimension. For each cornea, three areas on each of two different slides taken of representative areas of the flat-mount were counted, and the mean and standard deviation (SD) calculated.

Detection of IL-10 mRNA in transfected ovine corneas. Fresh corneas prepared as described above were infected with 4.5×10⁶ pfu Ad-mock or Ad-IL-10 for 2 hr or were incubated in medium without viral vector. They were then incubated in 3 ml complete medium at 32 C in air for 24 hr, after which a further 2 ml of complete medium was added and organ-culture was continued. At various time points thereafter, a central 8 mm diameter full-thickness disc of cornea was trephined and snap-frozen in liquid nitrogen. Each disc was pulverised in a pre-chilled stainless steel mortar and pestle. Total RNA was extracted with Total RNA Extraction Reagent (Advanced Biotechnologies Ltd., Surrey, UK), treated with DNAse (GlassMax MicroIsolation Kit, Life Technologies, Melbourne, VIC, Australia) and reverse-transcribed using a commercially-available first-strand cDNA synthesis kit (Amersham Pharmacia; UK Limited, Buckinghamshire, England) according to the manufacturers' recommendations. To control for residual ovine genomic or viral DNA contamination, samples were subjected to the same reverse-transcription step after inactivation of the reverse transcriptase at 95 C for 60 min. Dilutions of cDNA were amplified in 25 l total volume by PCR. The reaction mixture for IL-10 and -actin was 10 mM Tris-HCL (pH 8.3), 0.15M KCl (Perkin Elmer Roche Molecular Systems, Branchburg, N.J., USA), 0.2 mM of each dNTP (Amersham Pharmacia Biotech UK Limited, Buckinghamshire, England), 1.5 mM MgCl₂, 1 mM of each primer, 1 unit AmpliTaq-Gold (all from Perkin Elmer Roche Molecular Systems, Branchburg, N.J., USA) and 5 l of sample. The reaction buffer for glyceraldehyde 3-phosphate dehydrogenase (GAPDH) contained 2 mM MgCl₂ but was otherwise identical. Primer sequences amplified a 307 base pair region for IL-10 (SEQ. ID NO.1 5′-GCAGCTGTACCCACTTCCCA-3′, SEQ. ID NO.2 5′-AGAAAACGATGACAGCG-3′), a 317 base pair region for actin (SEQ. ID NO.3 5′-ATCATGTTTGAGACCTTCAA-3′, SEQ. ID NO.4 5′-CATCTCTTGCTCGAAGTCCA-3′), and a 527 base pair region for GAPDH (SEQ. ID NO.5 5′-ACCACCATGGAGAAGGCTGG-3′, SEQ. ID NO.6 5′-CTCAGTGTAGCCCAGGATGC-3′). After one cycle of 15 min at 94 C₁₋₄₀ cycles of amplification were performed, each consisting of annealing at 55 C for 30 sec, extension at 72 C for 30 sec and 94 C for 1 min, final extension at 55 C for 30 sec, 72 C for 20 sec and 35 C for 10 sec. Amplified products were electrophoresed on 1.5% agarose w/v gels.

Orthotopic corneal transplantation in sheep. Adult female Merino-cross breed sheep were acclimatised in groups of at least two animals for at least one week in indoor pens and were fed water ad libitum and chaff supplemented with lucerne hay. Twelve mm diameter penetrating corneal transplantation was performed as previously described (9) in the right eye only. Post-operative care and inspection were as previously described and every graft was examined at the slit-lamp each day. Groups of sheep received unmodified corneal grafts, corneas infected with Ad-mock, or corneas that had been infected with Ad-IL-10 or Ad-P40-IL-12 according to optimised procedures. The order in which sheep were grafted was random amongst all groups. Rejection was defined as reported previously (9). In several sheep with long-surviving corneal grafts, attempts were made to induce rejection by placement of 8-0 braided silk sutures into the graft under general anaesthetic, as an inflammatory stimulus. Approval for all experimentation was obtained from the institutional Animal Welfare Committee.

End-point histology of corneal allografts. Corneal tissue was fixed in buffered formalin, embedded in paraffin wax, cut at 8 m and stained with haematoxylin and eosin.

Immunoperoxidase staining of corneal allografts. Hybridoma culture supernatants containing mouse mAbs to sheep cell-surface determinants were obtained from the Department of Veterinary Science, University of Melbourne, Parkville, VIC, Australia and included: SBU 41.19, anti-MHC class I monomorphic epitope (23); SBU 28.1, anti-MHC class II monomorphic epitope (24); SBU 1-11-32, anti-CD45/leucocyte-common (unrestricted) antigen (25); SBU 44.38, anti-CD4 and SBU 38.65, anti-CD8 (26, 27); SBU 20.27, anti-CD1 (28); and SBU 72.87, anti-CD11a/LFA-1 (29). Culture supernatants from the hybridomas P3X63Ag8 (IgG1 isotype; European Collection of Animal Cell Cultures, Porton Down, Wiltshire, UK) and SAL5 (IgG2a isotype; gift of Dr L Ashman, IMVS, Adelaide, S A, Australia) were used as negative controls. Grafted eyes were harvested immediately post-mortem and the cornea excised, fixed, stained and scored as previously described (9).

Adenoviral antibody titres in fluids from sheep with corneal allografts. Immediately post-mortem, anterior chamber fluid was collected and snap-frozen at −80 C. Venous peripheral blood was also collected, the serum separated and similarly snap-frozen. Antibody titres to adenovirus were determined by a standard complement fixation test in the local reference laboratory using reagents from Biowhittaker Northfield Laboratories, Adelaide, S A, Australia.

Statistical analysis of data. Corneal graft survival data were analysed with the Mann-Whitney U-test, corrected for ties.

Results

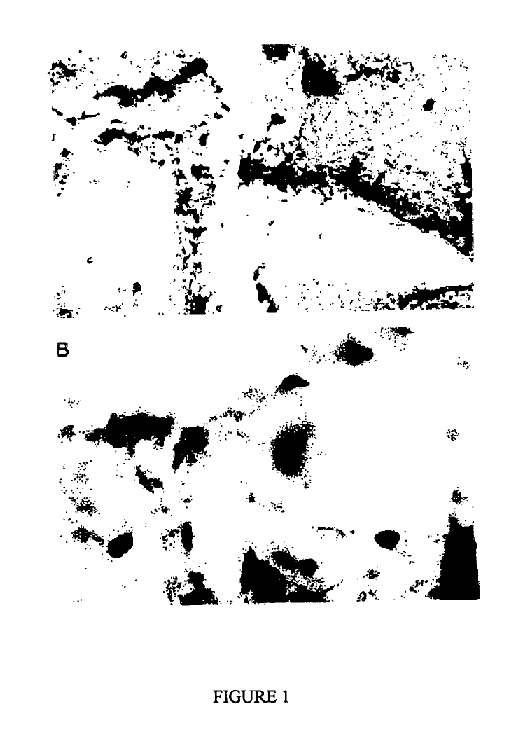

Replicative capacity of ovine corneal endothelium. Ovine corneal endothelium was deliberately injured and the corneas organ-cultured in the presence of ³H thymidine. The site of injury was still clearly visible at the light microscope on corneal endothelial flat-mounts harvested after 3 days in organ-culture. Uptake of ³H thymidine into the nuclei of the occasional endothelial cell close to the site of the injury was observed (FIG. 1A) and very rare mitotic Figures were identified (FIG. 1B). Uptake of ³H thymidine was limited to the close vicinity of the injury: no uptake occurred in the corneal periphery. Uninjured corneas (negative control) showed no uptake of ³H thymidine and corneas incubated with the epithelial surface in contact with isotope-containing solution (positive control) showed substantial uptake (not shown). The data suggested that the replicative capacity of ovine corneal endothelium was very limited and that, for example, replication-deficient adenoviral virus (which remains episomal) would thus be a suitable vector for gene transfer to ovine endothelium.

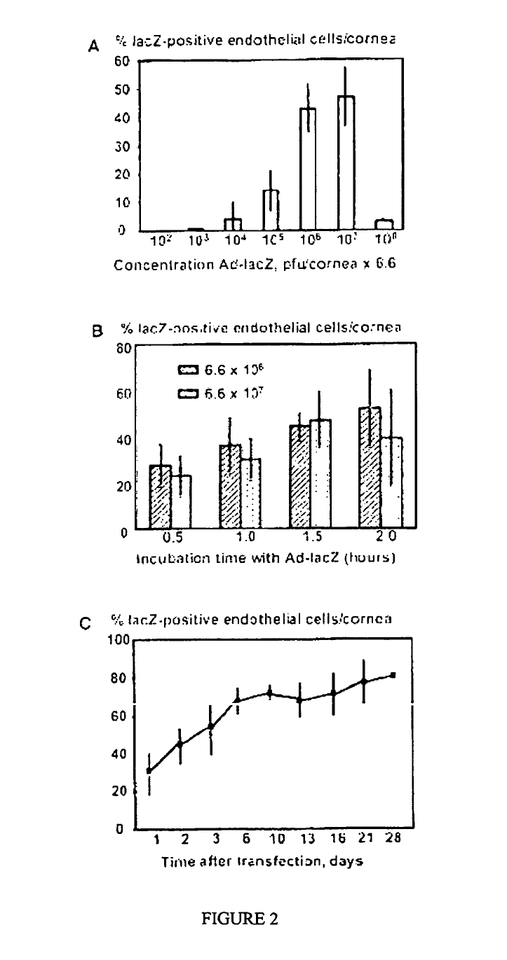

Adenoviral-mediated reporter gene transfer to ovine corneal endothelium. Ovine corneas were transfected in vitro with Ad-lacZ. At 6.6×10²-6.6×10⁴ pfu/cornea, single-galactosidase-positive cells were observed scattered over the endothelial monolayer. Increasing the virus concentration increased the number of -galactosidase-positive cells to a maximum of approximately 50% (FIG. 2A), although a drop in expression was observed at 6.6×10⁸ pfu/cornea. A concentration of 6.6×10⁶⁻⁷ pfu/cornea was judged to yield optimal expression. None of the negative controls (no virus applied, Ad-mock applied) showed expression of -galactosidase at any time. Reporter gene expression was observed only in corneal endothelium, not in stromal keratocytes. No visible toxic effects on the cornea were observed at any virus concentration. The influence of varying the time that the vector was in contact with corneal endothelium was investigated at 6.6×10⁶ and 6.6×10⁷ pfu/cornea (FIG. 2B): about 30% of cells were infected within the first hour, the number of positive cells increasing to about 50% at 2 hours. Duration of reporter gene expression was examined in a time-course experiment using 6.6×10⁷ pfu/cornea and an infection time of 1.5 hr: 30% cells expressed-galactosidase after 24 hr, rising to approximately 70% at day 6, and expression-remained at this level for the 28-day observation period (FIG. 2C).

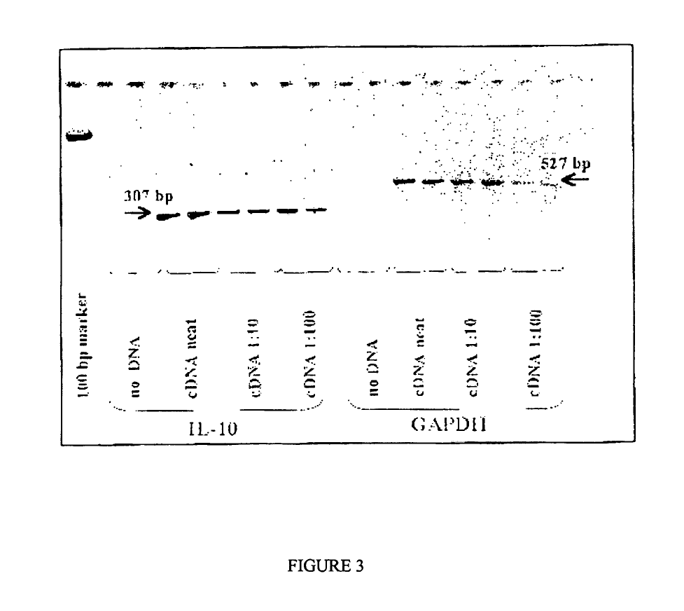

Detection of IL-10 mRNA in IL-10 gene-modified organ-cultured ovine corneas. Ad-IL-10 was used to transfer the gene encoding ovine IL-10 into sheep corneal endothelium using conditions optimised for reporter gene expression, and the corneas were cultured in vitro for up to 21 days. Reverse transcription PCR was used to detect presence of mRNA for IL-10; -actin and GAPDH served as housekeeping controls. No amplification of genomic or adenoviral IL-10 was observed in controls in which the reverse transcriptase had been inactivated after DNAse-I treatment of the isolated RNA preparations. Specific mRNA for ovine IL-10 was observed 24 hr after adenoviral infection and at various time points thereafter (Table 1), and could still be detected after corneas had been organ-cultured for 21 days (FIG. 3).

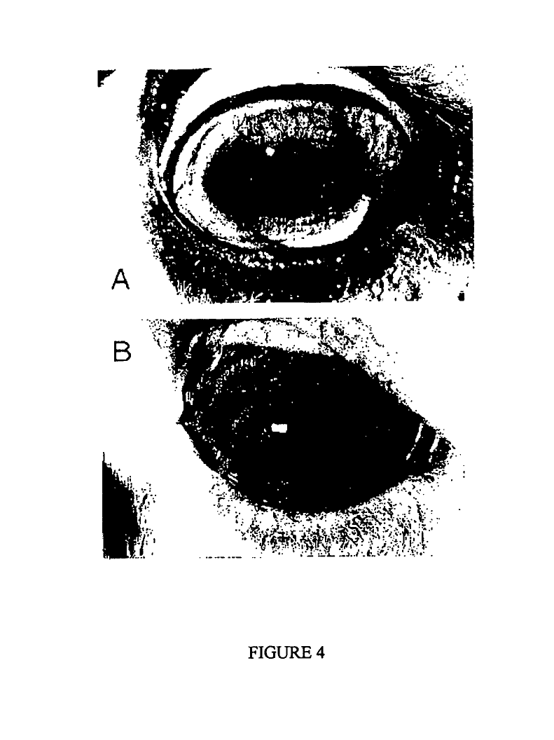

Orthotopic transplantation of gene-modified donor corneas in outbred sheep. The Ad-IL-10 and Ad-P40-IL-12 vectors were used to infect corneal endothelium of donor corneas immediately prior to orthotopic corneal transplantation in outbred sheep (FIG. 4). Controls included unmodified donor corneas and corneas infected with Ad-mock. Corneal graft survival data are shown in Table 2: corneal grafts modified by insertion of the gene encoding IL-10 into the donor endothelium survived significantly longer than unmodified controls (p=0.019) or the combined unmodified and mock virus-infected control groups (p=0.011). There was no difference in the time at which host vessels crossed the graft-host-junction amongst the groups (p>0.05). Longer survival was also demonstrated in corneas modified by c-section of the genes encoding the P-40 subunit of IL-12 (median 45 days). Post-operative inflammation was no more severe, and lasted for no longer, in the groups receiving adenovirus-treated corneas compared with the controls. End-point histology in sheep that showed clinical rejection of their grafts showed a similar picture in all instances: there was no difference amongst the experimental groups. Similarly, immunoperoxidase staining showed that rejecting gene-modified corneas contained a cellular infiltrate similar to that seen in rejecting unmodified or mock virus-infected grafts, with a substantial infiltrate of both CD4-positive and CD8-positive cells. No antibody to adenovirus was detectable in anterior chamber fluid or serum from 2 sheep at post-mortem.

Attempts were made to induce rejection in two sheep from the IL-10 group with gene-modified, long-surviving (>150 days) corneal grafts by placement of silk sutures into the graft at 196 and 303 days post-graft, respectively. In both cases, inflammation of the graft ensued and rejection occurred within two weeks, indicating that neither recipient was tolerant of the graft. Immunohistochemistry indicated that the infiltrate in these rejected grafts was similar to that seen in unmodified grafts.

Discussion

³H thymidine is incorporated into DNA by proliferating cells and can be visualised by autoradiography. This method has been used previously (30-33) to investigate the mitotic activity of corneal endothelium in many species. In the sheep cornea, uninjured endothelia incubated with ³H thymidine did not take up the isotope. Localized uptake into single endothelial cells was observed after deliberate injury, but proliferation was insufficient to cover the defect over three days. Our data suggest that ovine corneal endothelium has a very limited mitotic potential: some proliferation can be induced by a triggering event, but does not otherwise occur. For practical purposes, then, ovine corneal endothelium may be considered essentially amitotic. This resembles the situation in human and cat, where little mitosis occurs and defects are mainly covered by gradual sliding and enlargement of existing cells (30-33).

Replication-deficient adenoviruses, which remain episomal and do not integrate into the host genome, are suitable vectors for gene therapy of amitotic cells. A replication-defective adenovirus proved an efficient vector for gene transfer to 70-80% of ovine corneal endothelial cells. Optimal expression of the reporter gene in vitro was obtained with 6.6×10⁶ pfu per cornea. Given that the sheep cornea contains approximately 8×10⁵ endothelial cells, 6.6×10⁶ pfu represents a multiplicity of infection of >10 virions per cell. Infection at higher concentrations of the vector was less efficient, but no obvious toxic effects were apparent at any viral concentration. The optimal concentration was similar to that found by other authors for infection of rabbit corneal endothelium with adenoviral vectors (13-15). Other authors have observed reporter gene expression in 7% of rabbit corneal endothelial cells using Lipofectamine (14), and in a relatively small proportion of bovine endothelial cells using dioleoyl phosphatidylethylanolamine (34). More recently, George and his colleagues have demonstrated that activated polyamidoamine dendrimers, a novel class of non-viral agent, can successfully be used to transfer a gene into 6-10% of rabbit and human corneal endothelial cells (35). Adenovirus, however, appears significantly more efficient in achieving gene transfer than are non-viral agents.

Adenovirus binds to surface receptors and enters the cell by endocytosis via clathrin-coated vesicles, a fast process (36, 37). DNA replication in replicative adenoviruses starts approximately 8 hours after infection (38). In the sheep cornea, most adenoviral infection occurred within the first hour, but a delay of 5-6 days was observed before reporter gene expression was maximal. A time lag of 3-7 days for lacZ expression driven by either the CMV or RSV promoter has been observed after adenoviral transfer to human corneal endothelium (12, 14, 15). We observed lacZ expression in the sheep corneal endothelium to be stable for 4 weeks in vitro. Investigators working in the rabbit have found expression of lacZ for 3-4 weeks in in vitro experiments (38), but for only 1-2 weeks after orthotopic corneal transplantation (10). The protein-galactosidase has a half-life of 2 weeks in neurons (39) but shorter expression has been observed in other tissues such as respiratory epithelium (40), possibly due to gene silencing by promoter extinction (41). In the absence of a monoclonal antibody specific for ovine IL-10, expression of IL-10 product in ovine corneal endothelial cells was assessed indirectly by detection of mRNA for IL-10 in transfected, organ-cultured corneas. We were able to detect IL-10 mRNA in ovine corneal endothelium for at least 3 weeks in vitro.

In the absence of immunosuppression, corneal graft rejection in the sheep occurs at approximately three weeks post-operatively (9). We therefore reasoned that expression of a transferred gene encoding an immunomodulatory cytokine for 3-4 weeks in vivo might be sufficient to modulate graft rejection. Inflammatory responses in the eye following reporter gene transfer have been reported previously (13), and similar findings have been observed in other immunoprivileged tissues such as brain (42). However, we considered it possible that expression of an immunomodulatory cytokine in a privileged site such as the eye might be sufficient to ameliorate any local immune responses to the viral vector, as well as to the allograft. Interestingly, Qin and colleagues have previously reported modulation of the immune response to both alloantigen and adenovirus antigens in a murine cardiac allograft model following adenoviral-mediated gene therapy with viral IL-10 (43).

Obvious toxicity that could be attributed to the use of the adenoviral vector was absent. The adenoviral construct used to deliver the target gene to donor corneal endothelium did not elicit a measurable antibody response in the sheep after corneal transplantation, and did not induce noticeable ocular inflammation over the time-course of the experiment. Host vessels extended from the limbus towards all corneal grafts at the same rate, irrespective of the experimental group. In most animals that received gene-modified donor corneas, neovascularization was not accompanied by corneal graft rejection and the corneal vessels in these sheep did not maintain patency.

In our experiments, gene transfer of IL-10 to the donor cornea immediately prior to transplantation prolonged corneal allograft survival to a significant extent in the cohort as a whole. In two cases, graft survival was prolonged indefinitely (>150 days). Allograft survival was also prolonged in the group where corneal tissue was modified with P-40 of IL-12. It is notable that these result were obtained without the use of any other immunosuppressive therapy at all and in particular, without use of topical glucocorticosteroid. However, some sheep with IL-10-modified donor corneas rejected their grafts within the same time-frame as did the control animals. In these animals, graft rejection was indistinguishable at either a macroscopic or microscopic level from that observed in the controls. In particular, the extent and composition of the leucocytic infiltrate was similar in all cases and there was no obvious difference in expression of MHC class I or II molecules within the graft. That tolerance was not induced in the long-survivors is evinced by the observation that these animals did reject their grafts after deliberate application of an inflammatory stimulus to the graft. We hypothesize that expression of IL-10 by corneal endothelium was sufficient to modulate or significantly delay rejection in the majority of animals, but that rejection overwhelmed the immunomodulation in a minority of recipients.

Both cellular (44-46) and viral IL-10 (43, 47-49) have been reported to prolong allograft survival and to modulate chronic rejection in a variety of small animal models, and IL-10 gene knock-out mice show decreased cardiac allograft survival and increased evidence of chronic rejection (50, 51). However, in at least one report, systemic administration of murine IL-10 was shown to exacerbate murine cardiac allograft rejection (52), and further, subconjunctival and systemic administration of various doses of murine IL-10 has been shown to be ineffective in prolonging corneal graft survival in the rat (53). The effect of IL-10 on allograft survival appears to be dependent upon both timing of administration (45) and upon dose (46). Mouse and human IL-10 may not be entirely homologous with respect to function: in particular, the former can be immunostimulatory for murine T cells (16), a property not shared by human IL-10 or viral IL-10, although it has been suggested informally that endotoxin levels in various cytokine preparations may have affected some results. Ovine IL-10 has been shown to inhibit inflammatory cytokine production by sheep macrophages (54) and we believe it may have functional properties akin to human IL-10.

In summary, we report that delivery of genes encoding an immunomodulatory cytokines, mammalian IL-10 and P-40 subunit of IL-12, into donor corneal endothelium prior to transplantation results in significant prolongation of corneal allograft survival in an outbred model in which the endothelium is essentially non-replicative, and in which rejection appears very similar to human corneal graft rejection at both clinical and histological levels.

Similar techniques to those adopted in delivering genes encoding immunomodulatory cytokines to cornea tissue can be adopted in delivery of genes encoding immunoglobulins or immunoglobulin fragments to ocular tissue, as shown below.

### EXAMPLE 2

### Prolongation of Rat Corneal Allograft Survival by Gene Transfer-Mediated Intraocular Expression of Anti-CD28

Corneal transplantation is a well-accepted treatment for sight-threatening corneal opacification, but some grafts fail. The main cause of corneal graft failure is irreversible rejection, a T cell-dependent process.

Aim

To determine whether gene transfer of cDNAs encoding engineered antibody fragments reactive with T cell costimulatory molecules to donor corneas will prolong rat corneal allograft survival.

Methods

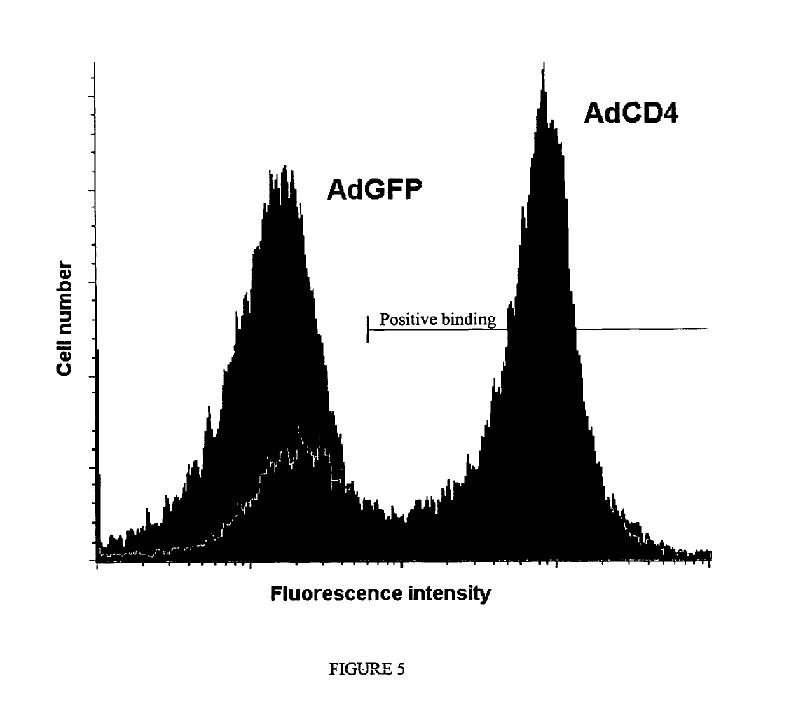

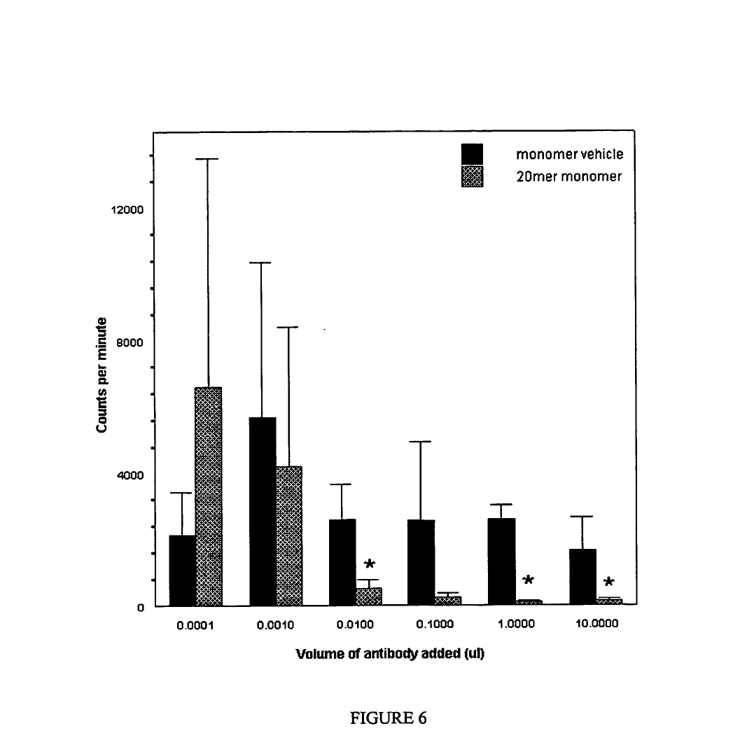

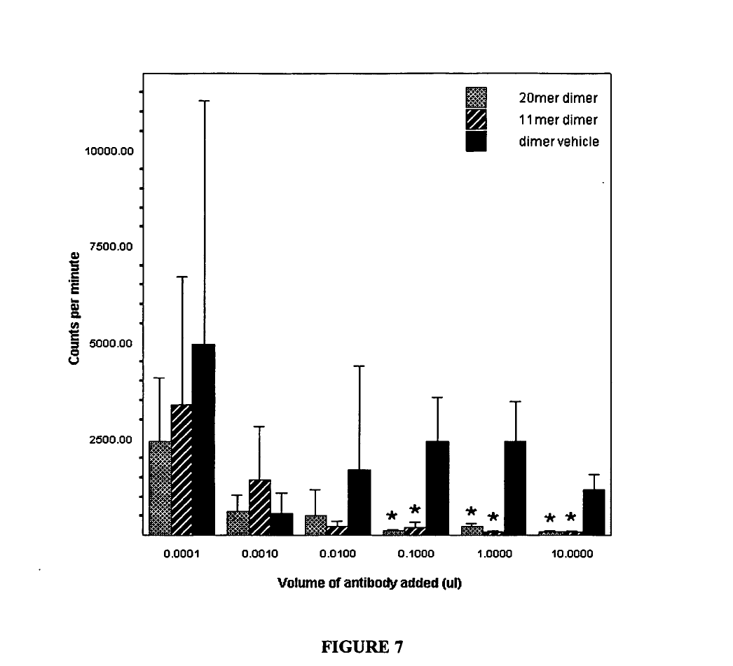

Replication-deficient adenoviruses encoding the green fluorescent protein (eGFP) reporter gene and secretory single-chain variable-domain antibody fragments (scFv) against rat CD4 (Ad-CD4) or rat CD28 (Ad-CD28) were constructed and characterised. Adenovirus-infected corneas were examined by reverse transcription-PCR to detect expression of specific mRNAs and by fluorescence microscopy to detect expression of eGFP. Functional scFv proteins were detected by flow cytometry on rat thymocytes and CD4+ transfectants (FIG. 5). The ability of scFvs to block allostimulation was measured in a one-way mixed lymphocyte reaction (MLR) (FIGS. 6, 7). Donor Wistar-Furth rat corneas were infected with adenoviruses ex vivo as described below, immediately prior to orthotopic transplantation into Fischer 344 recipients and graft survival was determined by daily microscopic examination and endpoint histology.

In Vitro Transduction of Rat Corneas with Replication-Deficient Adenoviral Vectors

The donor rat was killed by overdose of inhalation anaesthetic (halothane). Eyes were removed and decontaminated in 10% w/v povidone-iodine (Faulding Pharmaceuticals, Salisbury, Australia) for 2 minutes, before being rinsed twice in balanced salt solution (BSS: Cytosol Ophthalmics. Lenoir, N.C.) Under asceptic conditions corneas were dissected with a 1-2 mm scleral rim. The iris was removed and the corneas placed in HEPES-buffered RPMI 1640 medium (ICN-Pharmaceuticals, Costa Mesa, Calif.) supplemented with 2% volume/volume (v/v) heat inactivated (56° C., 30 min) FCS, 100 IU/ml penicillin, 100 μg/ml streptomycin sulfate and 2 mM L-glutamine (all from Gibco BRL, Gaithersburg, Md.). Corneas were transferred, endothelium up, to sterile 96-well round bottomed plates (Nalge Nunc International, Rochester, N.Y.) and transduced with adenoviral vectors diluted in HEPES-buffered RPMI 1640 medium supplemented with 2% FCS for 2 hours at 37° C. in 5% CO2 in air in a total volume of 100 μl. Corneas were used for grafts or placed in 2 ml HEPES-buffered RPMI 1640 medium supplemented with 10% FCS plus 2.5 μg/ml amphotericin B (Gibco BRL, Gaithersburg, Md.) at 37° C. in 5% CO₂ in air for up to 7 days.

Results

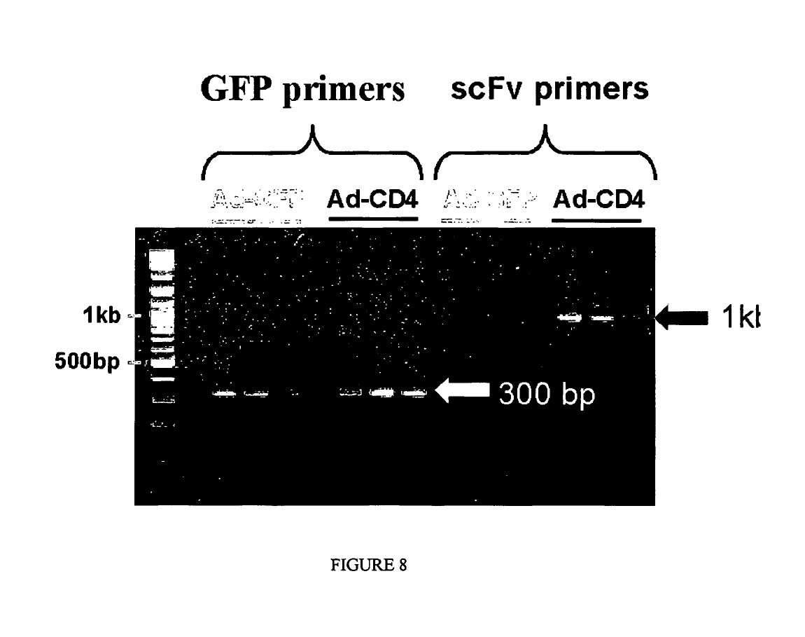

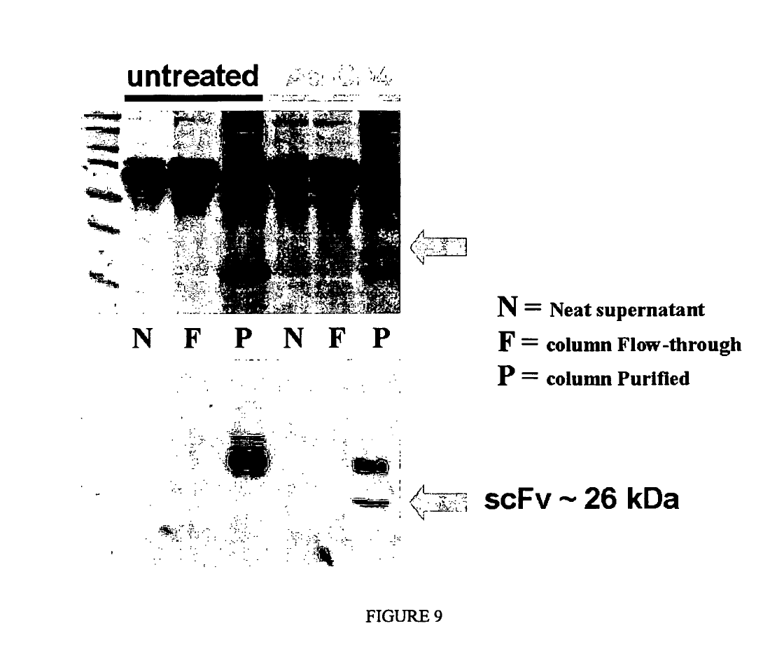

Viral loads of 5×10⁷ pfu/cornea produced eGFP expression in up to 95% of corneal endothelial cells after 4 days of corneal organ-culture. Specific scFv mRNAs were detectable in corneal tissue (FIG. 8) and secreted anti-CD4 scFv (FIG. 9) and anti-CD28 scFv were quantifiable in culture supernatants from 2 days post-infection. Anti-rat CD4 scFv blocked proliferation in MLR. Mock-treated corneal allografts (n=12) rejected at a median of day 12 after graft compared with day 11 for Ad-CD4-treated allografts (n=10; p=0.46 cf. controls) and day 16 for Ad-CD28-treated allografts (n=10; p=0.026 cf. controls); two recipients in the Ad-CD28-treated group exhibited prolonged graft survival of >60 days.

### CONCLUSION

Expression of anti-CD28 antibody fragments by donor corneas can significantly prolong corneal allograft survival.

The present invention has been described by way of example only and it should be recognised that modifications and/or alterations to the specific aspects of the invention which would be apparent to persons skilled in the art based on the disclosure herein, are also considered to fall within the spirit and scope of the invention.

TABLE 1
Detection of mRNAs for ovine IL-10 or housekeeping
genes -actin and GAPDH in ovine corneas after
infection with adenoviral vectors^{a}

| | | Corneas infected with:^{a} | | |
|---|---|---|---|---|
| Time after | Primer | medium | | |
| infection | identity | control | Ad-mock^{b} | Ad-IL-10^{c} |
| 2 hours | IL-10- | −^{d} | − | (+)^{d} |
| | actin/GAPDH | +^{d} | + | + |
| 3 days | IL-10- | − | − | + |
| | actin/GAPDH | + | + | + |
| 7 days | IL-10- | − | − | + |
| | actin/GAPDH | + | + | + |
| 10 days | IL-10- | NT^{e} | − | + |
| | actin/GAPDH | NT | + | + |
| 14 days | IL-10- | NT | − | + |
| | actin/GAPDH | NT | + | + |
| | IL-10- | NT | − | |
| | actin/GAPDH | NT | + | + |

^{a}For each primer at each time-point, 1-3 individual corneas were examined;
^{b}Ad-mock, replication deficient E1, E3-deleted-adenovirus-type 5 containing an empty plasmid;
^{c}Ad-IL-10, replication deficient E1, E3-deleted adenovirus type 5 encoding full-length ovine IL-10;
^{d}−represents no signal detected by PCR, (+)represents a weak positive signal detectable only in an undiluted cDNA sample, +represents a strong positive signal;
^{e}NT, not tested.

TABLE 2
Survival of control and gene-modified orthotopic corneal grafts in outbred
sheep transplanted with unmodified donor corneas, or with corneas transfected
before transplantation with Ad-mock, or with corneas transfected before
transplantation with Ad-IL-10, Ad-P40-IL-12 or Ad-I1-4

| | | Day vessels | |
|---|---|---|---|
| Donor cornea | n | crossed into graft^{a} | Day of rejection |
| Unmodified | 7 | 11, 10, 9, 8, 10, 9, 9 | 18, 19, 19, 20, 20, 22, 32 |
| | | median = 9 | median = 20 |
| Mock-transfected | 3 | 5, 7, 8 | 19, 21, 29 |
| | | median = 7 | median = 21 |
| IL-10-transfected | 9 | 5, 9, 9, 10, 10, 9, 11, 11, 9 | 19, 20, 30, 33, 55, 66, 88, |
| | | median = 9 | >196, >300^{b} |
| | | | median = 55 |
| P-40 IL-12 transfected | 9 | — | 22, 23, 32, 36, 45, >50, 93, 93, |
| | | | >100 |
| | | | median = 45 |

^{a}For each recipient sheep, the day post-graft at which corneal blood vessels crossed from the recipient corneal edge into the graft is shown, together with the day post-graft at which the graft was deemed to have undergone rejection. Individual recipients in columns 3 and 4 are listed in the same order;
^{b}p = 0.019 compared with unmodified controls, p = 0.011 compared with combined control groups (Mann-Whitney test, two-tailed).

### REFERENCES

1. Williams K A, Muehlberg S M, Lewis R F, Coster D J on behalf of all contributors to the Australian Corneal Graft Registry (ACGR). How successful is corneal transplantation? A report from the Australian Corneal Graft Register. Eye 1995; 9: 219.
2. Griffith T S, Ferguson T A. The role of FasL-induced apoptosis in immune privilege. Immunology Today 1997; 18: 240.
3. Williams K A, Coster D J. Clinical and experimental aspects of corneal transplantation. Transplantation Reviews 1993; 7: 44.
4. Pepose J S, Gardner K M, Nestor M S, Foos R Y, Pettit T H. Detection of HLA class I and II antigens in rejected human corneal allografts. Opthalmology 1985; 92: 1480.
5. Holland E J, Chan C-C, Wetzig R P, Palestine A G, Nussenblatt R B. Clinical and immunohistologic studies of corneal rejection in the rat penetrating keratoplasty model. Cornea 1991; 10: 374.
6. Whitcup S M, Nussenblatt R B, Price F W, Chan C-C. Expression of cell adhesion molecules in corneal graft rejection. Cornea 1993; 12: 475.
7. Sano Y, Osawa H, Sotozono C, Kinoshita S. Cytokine expression during orthotopic corneal allograft rejection in mice. Invest Opthalmol V is Sci 1998; 39: 1953.
8. Tuft S J, Coster D J. The corneal endothelium. Eye 1990; 4:3 89.
9. Williams K A, Standfield S D, Mills R A, et al. A new model of orthotopic penetrating corneal transplantation in the sheep: graft survival, phenotypes of infiltrating cells and local cytokine production. Aust N Z J Opthalmol 1999; 27: 127.
10. Larkin D F, Oral H B, Ring C J, Lemoine N R, George A J. Adenovirus-mediated gene delivery to the corneal endothelium. Transplantation 1996; 613: 363.
11. Borras T, Tamm E R, Zigler J S Jr. Ocular adenovirus gene transfer varies in efficiency and inflammatory response. Invest Opthalmol V is Sci 1996; 37: 1282.
12. Budenz D L, Bennett J, Alonso L, Maguire A. In vivo gene transfer into murine corneal endothelial and trabecular meshwork cells. Invest Opthalmol V is Sci 1995; 36:2211.
13. Oral H B, Larkin D F, Fehervari Z, et al. Ex vivo adenovirus-mediated gene transfer and immunomodulatory protein production in human cornea. Gene Therapy 1997; 4: 639.
14. Arancibia-Carcamo C V, Oral H B, Haskard D O, Larkin D F, George A J. Lipoadenofection-mediated gene delivery to the corneal endothelium: prospects for modulating graft rejection. Transplantation 1998; 65: 62
15. Fehervari Z, Rayner S A, Oral H B, George A J, Larkin D F. Gene transfer to ex-vivo stored corneas. Cornea 1997; 16: 459.
16. De Waal Malefyt R, Yssel H, Roncarolo M-G, Spits H, de Vries J E. Interleukin-10. Current Opin Immunol 1992; 4: 314.
17. Moore K W, O Garra A, de Waal Malefyt R, Vieira P, Mosmann T R. Interleukin-10. Annu Rev. Immunol 1993; 11:165.
18. Graham F L, Prevec L. Manipulation of adenovirus vectors. In: Murray E J, ed Methods in Molecular Biology Gene Transfer and Expression Protocols, Vol. 7. Clifton, N.J.: The Humana Press, 1991: 109.
19. Graham F, Smiley J, Russell W C, Naim R. Characteristics of a human cell line transformed by DNA from human adenovirus type 5. J Gen Virol 1977; 36: 59.
20. Kanegae Y, Makimura M, Saito I. A simple and efficient method for purification of infectious recombinant adenovirus. Japn J Med Sci Biol 1994; 47: 157.
21. Lojda Z, Gossrau R, Schiebler T H. In: Enzymhistochemische Methoden. Berlin-Heidelberg: Springer Verlag, 1976: 128.
22. Sherrard E. The corneal endothelium in vivo: its response to mild trauma. Exp Eye Res. 1976; 22; 347.
23. Gogolin-Ewens K J, Mackay C R, Mercer W R, Brandon M R. Sheep lymphocyte antigens (OLA). I Major histocompatibility complex class I molecules. Immunology 1985:56:717.
24. Puri N K, Mackay C R, Brandon M R. Sheep lymphocyte antigens (OLA). II Major histocompatibility complex class II molecules. Immunology 1985; 56: 725.
25. Maddox J F, Mackay C R, Brandon M R. The sheep analogue of leucocyte common antigen (LCA). Immunology 1985; 55: 347.
26. Mackay C R, Maddox J F, Brandon M R. Surface antigens, SBU-14 and SBU-T8, of sheep T lymphocyte subsets defined by monoclonal antibodies. Immunology 1985; 55: 739.
27. Mackay C R, Maddox J F, Brandon M R. Three distinct subpopulations of sheep T lymphocytes. Eur J Immunol 1986:16:19.
28. Mackay C R, Maddox J F, Gogolin-Ewens K J, Brandon M R. Characterization of two sheep lymphocytic differentiation antigens, SBU-T1 and SBU-T6. Immunology 1085; 55: 729.
29. Grooby W L, Carter J K, Rao M M, et al. Use of an anti-LFA-1 antibody allograft rejection in sheep. Transplant. Proc 1992; 24: 2304.
30. Van Horn D L, Sendele D D, Seideman S, Buco P J. Regenerative capacity of the corneal endothelium in rabbit and cat. Invest Opthalmol V is Sci 1977; 16: 597.
31. Simonsen A H, Sorensen K E, Sperling S. Thymidine incorporation by human endothelium during organ culture. Acta Opthalmol 1981; 59: 110.
32. Tuft S J, Williams K A, Coster D J. Endothelial repair in the rat cornea. Invest Opthalmol V is Sci 1986; 27: 1199.
33. Treffers W F. 1982. Corneal endothelial wound healing. Nijmegen: Janssen Print, 1982.
34. Pleyer U, Druegg A, Bertelmann E, et al. Liposome-mediated gene transfer into corneal endothelial cells. Invest Opthalmol V is Sci 1997: 38: S402.
35. Hudde T, Rayner S A, Corner R M, et al. Activated polyamidoamine dendrimers, a non-viral vector for gene transfer to the corneal endothelium. Gene Therapy 1999; 6: 939.
36. Philipson L, Longberg Helm, K, Patterson U. Virus-Receptor interaction in an adenovirus system. J Virol 1968; 2: 1064.
37. Seth P, Fitzgerald D, H, Willingham M, Pastan I. Evidence that the penton base of adenovirus is involved in potentiation of toxicity of Pseudomonas exotoxin conjugated to epidermal growth factor. Mol Cell Biol 1984; 4: 1528.
38. Hoffman L M, Maguire A M, Bennett J. Cell-mediated immune response and stability of intraocular transgene expression after adenovirus-mediated delivery. Invest Opthalmol V is Sci 1997; 38: 2224.
39. Bloom D C, Maidment N T, Tan A, Dissette V B, Feldman L T, Stevens J G. Long term expression of a reporter gene from latent herpes simplex virus in the rat hippocampus. Brain Res Mol Brain Research 1995; 31: 48.
40. Goldman M J, Litzky L A, Engelhardt J F, Wilson J M. Transfer of the CFTR gene to the lung of nonhuman primates with E1-deleted, E2a-defective recombinant adenoviruses: a preclinical toxicology study. Hum Gene Therapy 1995; 6: 839.
41. Palmer T D, Rosman G J, Osborne W R, Miller A D. Genetically modified skin fibroblasts persist long after transplantation but gradually inactivate introduced genes. Proc Natl Acad Sci USA 1991; 88: 1330.
42. Byrnes A P, Rusby J E, Wood M J, Charlton H M. Adenovirus gene transfer causes inflammation in the brain. Neuroscience 1995; 66: 1015.
43. Qin L, Ding Y, Pahud D R, Robson N D, Shaked A, Bromberg J S. Adenovirus-mediated gene transfer of viral interleukin-10 inhibits the immune response to both alloantigen and adenoviral antigen. Hum Gene Ther 1997; 8: 1365.
44. Shinozaki K, Yahata H, Tanji H, Sakaguchi T, Ito H, Dohi K. Allograft transduction of IL-10 prolongs survival following orthotopic liver transplantation. Gene Therapy 1999; 6: 816.
45. Boehler A, Chamberlain D, Xing Z, et al. Adenovirus-mediated interleukin-10 gene transfer inhibits post-transplant fibrous airway obliteration in an animal model of bronchiolitis obliterans. Hum Gene Therapy 1998; 9: 541.
46. Zou X M, Yagihashi A, Hirata K, et al. Downregulation of cytokine-induced neutrophil chemoattractant and prolongation of rat liver allograft survival by interleukin-10. Surg Today 1998; 28:184.
47. Qin L. Chavin K D, Ding Y, et al Retrovirus-mediated transfer of viral IL-10 prolongs murine cardiac allograft survival. J Immunol 1996; 156: 2316.
48. DeBruyne L A, Li K, Chan S Y, Bishop D K, Bromberg J S. Lipid-mediated gene transfer of viral IL-10 prolongs vascularized cardiac allograft survival by inhibiting donor-specific cellular and humoral immune responses. Gene Therapy 1998; 5: 1079.
49. Brauner R, Nonoyama M, Laks H, et al. Intracoronary adenovirus-mediated transfer of immunosuppressive cytokine genes prolongs allograft survival. J Thorac Cardiovasc Surg 1997; 114: 923.
50. Raisanen-Sokolowski A, Mottram P I, Glysing-Jensen T, Satoskar A, Russell M E. Heart transplants in interferon-, interleukin 4, and interleukin 10 knockout mice. Recipient environment alters graft rejection. J Clin Invest 1997; 100:2449.
51. Räisänen-Sokolowski A, Glysing-Jensen T, Russell M E. Leukocyte-suppressing influences of interleukin (IL)-10 in cardiac allografts. Insights from IL-10 knockout mice. Am J Pathol 1998; 153: 1491.
52. Qian S, Li W, Li Y, et al. Systemic administration of cellular interleukin-10 can exacerbate cardiac allograft rejection in mice. Transplantation 1996; 62: 1709.
53. Torres P F, de Vos A F, Martins B, Kijlstra A. Interleukin 10 treatment does not prolong experimental corneal allograft survival. Ophthalmic Res 1999 31:2 97. Martin H M, Nash A D, Andrews
54. Borras; 2003, Exp. Eye Res. 76 643-52
55. Mandava et al Invest. Opthalmol. Vis. Sci. 2002 43 3338-48
56. Zhang et al Mol. Vis. 2003 9 465-72
57. Berdugo et al Antisense nucleic acid drug. dev. 2003 13 107-14
58. Kurz Opthalmol Clin. North Am 2002 15 405-10
59. Shestopolov et al, Exp. Eye Res. 2002 74 639-49
60. Grobhans Funct. Integr. Genomics 2000 1 142-5
61. Wolf and Jenkins Int. J. Oncol. 2002 21 461-68
62. Swift G J, Aggarwal R K, Davis G J, Coster D J, Williams K A. Survival of rabbit limbal stem cell allografts Transplantation 1996; 62: 568-574.
63. Williams K A, Brereton H M, Aggarwal R, Sykes P J, Turner D R, Russ G R, Coster D J. Use of DNA polymorphisms and the polymerase chain reaction to examine the survival of a human limbal stem cell allograft. Am J Opthalmol 1995; 120: 342-350.
64. Henderson T R, Coster D J, Williams K A. The long-term outcome of limbal allografts—the search for surviving cells. Br J Opthalmol 2001; 85: 604-609.
65. Mills R A D, Coster D J, Williams K A. Effect of immunosuppression on outcome measures in a model of rat limbal transplantation. Invest Opthalmol V is Sci 2002; 43: 647-655.

1. A method of modifying cells of ocular tissue to produce an immunoglobulin or immunoglobulin fragment of interest, which comprises exposing ocular tissue to an effective concentration for transfection of an expression vector comprising a nucleotide sequence encoding for the immunoglobulin or immunoglobulin fragment, for a period sufficient to allow transfection, such that cells of said ocular tissue produce the immunoglobulin or immunoglobulin fragment.

2.-72. (canceled)
